(19) United States
(12) Patent Application Publication
(10) Pub. No.: US 2025/0078673 A1
(43) Pub. Date: Mar. 6, 2025

(54) INFORMATION PROCESSING SYSTEM, INFORMATION PROCESSING METHOD, AND NON-TRANSITORY COMPUTER-READABLE MEDIUM STORING PROGRAM

(71) Applicant: CureApp, Inc., Tokyo (JP)

(72) Inventors: Fumi HISAKI, Tokyo (JP); Shinya FUTAGAMI, Tokyo (JP)

(73) Assignee: CureApp, Inc., Tokyo (JP)

(21) Appl. No.: 18/814,839

(22) Filed: Aug. 26, 2024

(30) Foreign Application Priority Data
Aug. 29, 2023 (JP) 2023-139116

(51) Int. Cl. G09B 5/02; A61B 5/00

(52) U.S. Cl. CPC G09B 5/02; A61B 5/021; G16H 20/70; A61B 5/7465

(57) ABSTRACT

A technology to encourage a user to continue improving user's lifestyle at a time when a predetermined period of time elapses since the user starts using an application that supports improvement of lifestyle is provided. An information processing system includes at least one processor, wherein the at least one processor that causes the user to start using the application which supports improvement of the user's lifestyle over a set period of improvement, and displays a content which encourages the user to continue improving the user's lifestyle at a time when a predetermined period of time elapses since the user starts using the application.

### CROSS REFERENCE TO RELATED APPLICATIONS

This application is based on and claims priority under 35 USC § 119 to Japanese Patent Application No. 2023-139116 filed Aug. 29, 2023, the disclosure is incorporated herein by reference in its entirety.

### BACKGROUND

### Technical Field

The present invention relates to an information processing system, an information processing method, and a non-transitory computer-readable medium storing a program.

### Related Art

The Patent Literature 1 describes a lifestyle-related improvement proposal device which acquires measured biological information of a first user or lifestyle information representing the lifestyle of the first user, and when the acquired information meets the specified conditions and transmits a message suggesting lifestyle improvement to the user device used by a given user.

The Patent Literature 2 describes a health management system in which information terminals for patients or the like includes a measurement screen display means for displaying a measurement screen for measurement for preventing and treating lifestyle-related disease, a reception means for receiving a patient's measurement data from a measuring instrument, a display means for displaying the measurement data, a transmission means for transmitting the measurement data to the management server, a reception means for receiving an advice of the healthcare worker from the medical professional information terminal, and a display means for displaying the received advice.

### CITATION LIST

### Patent Literature

Patent Literature 1: Japanese Patent Application Laid-Open Publication No. 2020-160569
Patent Literature 2: Japanese Unexamined Patent Application Laid-Open Publication No. 2011-180817

It is conceivable to make suggestions regarding improvement of lifestyle based on user's environmental information, regardless of the period of time since the user starts using an application that supports improvement of lifestyle. However, if such a proposal is made, for example, when the user just starts using the application for a short period of time, the so-called “slump” may not occur, and the proposal may not be effective.

The purpose of the present disclosure is to encourage the user to continue improving the user's lifestyle at a time when a predetermined period of time elapses since the user starts using the application that supports improvement of lifestyle.

### SUMMARY

The present disclosure provides an information processing system including at least one processor, wherein the at least one processor cause user to start using an application which supports improvement of the user's lifestyle over a set period of improvement, and displays a content which encourages the user to continue improving the user's lifestyle at a time when a predetermined period of time elapses since the user starts using the application.

The time when a predetermined period of time elapses may be a period of time when continuation of improving the user's lifestyle is likely to be difficult.

The time when the predetermined period of time elapses may be a time when the user's motivation for his/her appropriate lifestyle lowers or a time prior to the time when the user's motivation lowers.

Plural periods of time may be set as the time when the predetermined period of time elapses, and the plural periods of time may be set in a second half of the set period of improvement more than a first half of the set period of improvement.

At least one processor may display the content if a predetermined condition is satisfied when the predetermined period of time elapses. Alternatively, the at least one processor may display a first content as the content, if a predetermined condition is satisfied when the predetermined period of time elapses, and display a second content as the content if the predetermined condition is not satisfied when the predetermined period of time elapses.

In that case, the predetermined may be a condition that the user does not perform a behavior which infers continuation of improving the user's lifestyle. In that case, the predetermined condition may be a condition that a rate of use of the application by the user lowers. Further, the predetermined condition may be a condition that a rate of performing a predetermined behavior by the user with respect to a target lowers. In addition, the predetermined condition may be a condition that a number of characters in a diary related to improvement of the user's lifestyle which the user inputs lowers.

In addition, the predetermined condition may be a condition that the user's measurement value does not satisfy reference which infers continuation of improving the user's lifestyle.

Further, the predetermined condition may be a condition that a period when an event which is likely to obstruct continuation of improving the user's lifestyle occurs is approaching.

Furthermore, the predetermined condition may be a condition that the user is in an environment where it is likely to be difficult for the user to continue improving the user's lifestyle.

The content may be configured to enable the user to input information related to improvement of the user's lifestyle. In that case, the information related to the improvement of the user's lifestyle may be notified to a terminal of a mentor for the improvement of the user's lifestyle.

The present disclosure also provides an information processing method including causing a user to start using an application which supports improvement of the user's lifestyle over a set period of improvement, and displaying a content which encourages the user to continue improving the user's lifestyle at a time when a predetermined period of time elapses since the user starts using the application.

The present disclosure also provides a non-transitory computer-readable medium storing a program that causes a computer to execute causing a user to start using an application which supports improvement of the user's lifestyle over a set period of improvement, and displaying a content which encourages the user to continue improving the user's lifestyle at a time when a predetermined period of time elapses since the user starts using the application.

According to the present disclosure, it is possible to encourage the user to continue improving the user's lifestyle at a time when a predetermined period of time elapses since the user starts using the application that supports improvement of lifestyle.

### BRIEF DESCRIPTION OF DRAWINGS

Exemplary embodiments of the present invention will be described in detail based on the following figures, wherein:

FIG. 1 shows a whole configuration example of a prescription digital therapeutic app operation system in the exemplary embodiments;

FIG. 2 shows a hardware configuration example of a prescription digital therapeutic app operation server in the exemplary embodiments;

FIG. 3 shows a hardware configuration example of a terminal device in the exemplary embodiments;

FIG. 4 shows an example of a normal home screen which a patient terminal displays;

FIG. 5 shows an example of the 12th week home screen which a patient terminal displays;

FIG. 6 shows an example of the 16th week home screen which a patient terminal displays;

FIG. 7 shows an example of the 18th week first home screen which a patient terminal displays;

FIG. 8 shows an example of the 18th week second home screen which a patient terminal displays;

FIG. 9 shows an example of the 20th week home screen which a patient terminal displays;

FIG. 10 shows a functional configuration example of a prescription digital therapeutic app operation server in the exemplary embodiments;

FIG. 11 shows a specific example of patient management information stored in the storage section;

FIG. 12 shows a specific example of patient state management information stored in the storage section;

FIG. 13 shows a specific example of display control information stored in the storage section;

FIG. 14 is a sequence diagram showing an example of operations of the prescription digital therapeutic app operation system when patients register various kinds of information by performing activation of the prescription digital therapeutic app;

FIG. 15 is a sequence diagram showing an example of operations of the prescription digital therapeutic app operation system when patients use the prescription digital therapeutic app after performing activation of the prescription digital therapeutic app;

### DETAILED DESCRIPTION

Hereinafter, exemplary embodiments will be described with reference to the accompanying drawings.

### Overview of the Present Embodiments

The present embodiments provide an information processing system that causes a user to start using an application which supports improvement of the user's lifestyle over a set period of improvement, and displays a content which encourages the user to continue improving the user's lifestyle at a time when a predetermined period of time elapses since the user starts using the application.

Herein, various applications are considered as the application that supports improvement of the user's lifestyle, however, hereinafter, an application whose purpose is therapy for high blood pressure, which a patient of high blood pressure uses (hereinafter referred to as “prescription digital therapeutic app”) is described as an example.

In addition, when the medical insurance system is applied to the prescription digital therapeutic app, the use of the prescription digital therapeutic app by the patient is allowed only during the period when the medical insurance system is applied (hereinafter referred to as “insurance medical therapy period”). Therefore, as a set period of improvement to support the improvement of the user's lifestyle, the insurance medical therapy period will be used as an example below.

Further, “system” may be configured with a single device or multiple devices. In the following, the system configured with the single device is described as an example of the information processing system, and prescription digital therapeutic app operation server which operates the prescription digital therapeutic app is described as an example of the single device.

Hereinafter, exemplary embodiments of the present disclosure will be described with reference to the drawings.

### <Whole Configuration of a Prescription Digital Therapeutic App Operation System>

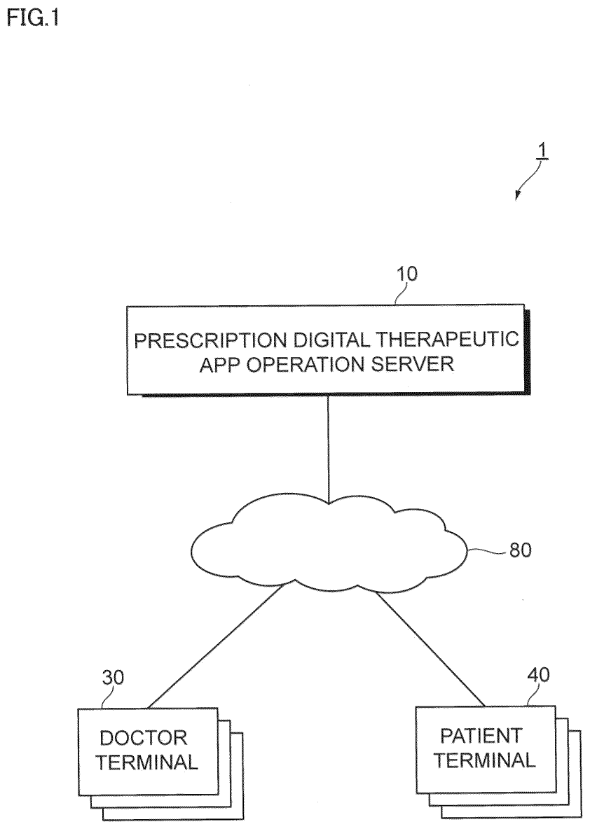

FIG. 1 shows a whole configuration example of a prescription digital therapeutic app operation system in the exemplary embodiments.

As shown, a prescription digital therapeutic app operation system 1 is configured with a prescription digital therapeutic app operation server 10, a doctor terminal 30 and a patient terminal 40 connected via a network 80.

The prescription digital therapeutic app operation server 10 is a server that executes distribution of prescription digital therapeutic app, verification of patients, storage of various information input through the prescription digital therapeutic apps, and provision of patient data to insurance medical institutions (specifically, doctor terminal 30). Note that the distribution of prescription digital therapeutic app may be performed by a business entity that is different from the entity that operates the prescription digital therapeutic app operation server 10.

Herein, it is assumed that the prescription digital therapeutic app is an application approved by the Pharmaceutical and Medical Device Act and patients who use the prescription digital therapeutic app can receive insurance medical therapy by a doctor during the insurance medical therapy period. Note that the insurance medical therapy here includes not only face-to-face medical therapy but also online medical therapy.

In the exemplary embodiments, the insurance medical therapy period is 6 months. However, 6 months is merely an example, and it may be 9 months or 12 months. In addition, it may also be defined in days, such as 180 or 360 days, or in weeks, such as after 26 or 52 weeks. Needless to say, these values are examples.

In the exemplary embodiments, the use of the prescription digital therapeutic app by the patient is allowed only during the insurance medical therapy period. In other words, it is possible to input and record blood pressure values and so on through the prescription digital therapeutic app.

The doctor terminal 30 is a terminal device that is operated by a doctor who prescribes the prescription digital therapeutic app in an insurance medical institution and so on. The plural terminals 30 are shown in FIG. 1. As the doctor terminal 30, for example, a desktop computer, a notebook type computer, a tablet type computer and a smartphone may be used. Alternatively, a wearable computer such as a smart glass or a head set may be used as the doctor terminal 30. Note that, within the allowable range, it is assumed that the doctor terminal 30 is operated by nurses, assistant nurses, pharmacists, public health nurses, nutritionists, hospital staffs and other medical personnel.

The patient terminal 40 is a terminal device that is operated by a patient who undergoes medical examination or therapy for high blood pressure. Plural patient terminals 40 are shown in FIG. 1. As the patient terminal 40, for example, a desktop computer, a notebook type computer, a tablet type computer and a smartphone may be used. Alternatively, a wearable computer such as a smart glass or a head set may be used as the patient terminal 40.

The network 80 is, for example, a local area network (LAN), the Internet, or a mobile communication system (4G, 5G).

FIG. 1 illustrates the case that the prescription digital therapeutic app operation server 10, the doctor terminal 30 and the patient terminal 40 are connected to one network 80, however, they may be connected via plural networks connected one another.

### <Hardware Configuration of the Prescription Digital Therapeutic App Operation Server>

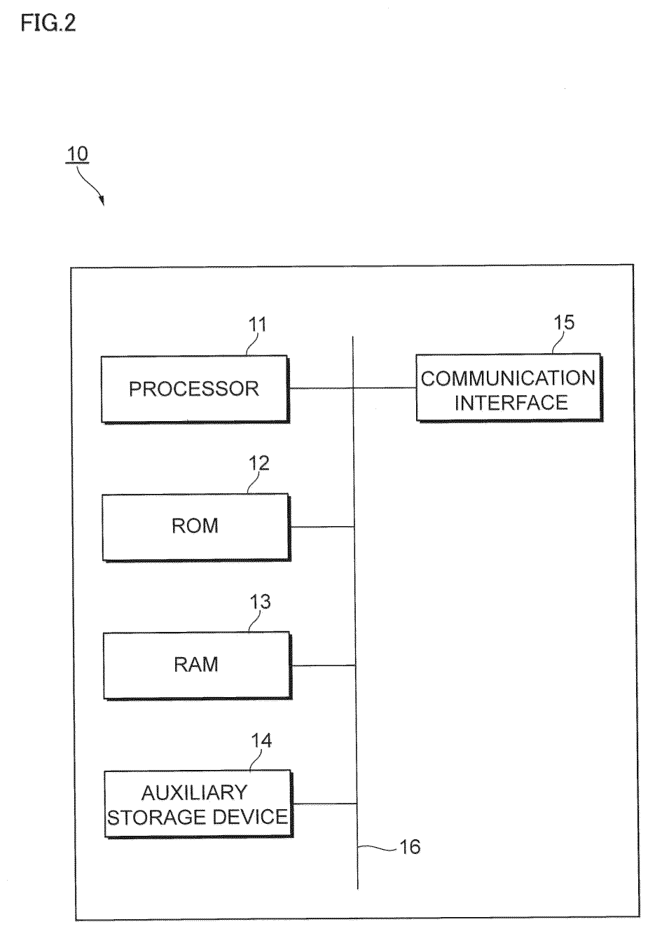

FIG. 2 shows a hardware configuration example of a prescription digital therapeutic app operation server 10 in the exemplary embodiments.

As shown, the prescription digital therapeutic app operation server 10 includes a processor 11, a Read Only Memory (ROM) 12, a Random Access Memory (RAM) 13, an auxiliary storage device 14, and a communication interface 15. Each device is connected via a bus and other signal wires 16.

The processor 11 is a device that executes various kinds of functions through the execution of programs. ROM 12 stores a Basic Input Output System (BIOS), etc. RAM 13 is used as a work area of the processor 11. The processor 11, ROM 12, and RAM 13 function as a computer.

The auxiliary storage device 14 is configured with, for example, a hard disk device or a semiconductor storage. The auxiliary storage device 14 stores data related to verification of a patient who is prescribed the prescription digital therapeutic app and patient data registered through the prescription digital therapeutic app in addition to the therapy application.

The communication interface 15 is an interface for communicating with other servers or terminal devices via the network 80. The communication interface 15 complies with Ethernet (registered trademark), Wi-Fi (registered trademark), mobile communication systems and other communication standards.

### <Hardware Configuration of Terminal Device>

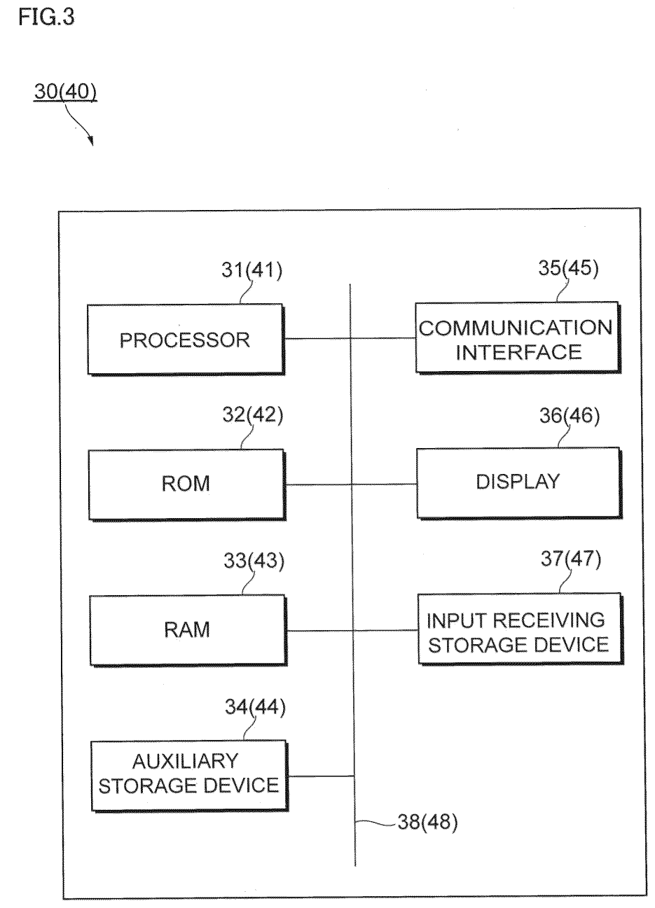

FIG. 3 shows a hardware configuration example of a terminal device in the exemplary embodiments. The hardware configuration shown in FIG. 3 is in common with the doctor terminal 30 and the patient terminal 40. Therefore, hereinafter, the patient terminal 40 is described as a representative example of a terminal device.

As shown, the patient terminal 40 includes a processor 41, a ROM 42, a RAM 43, an auxiliary storage device 44, a communication interface 45, a display 46 and an input receiving device 47. Each device is connected via a bus and other signal wires 48.

The processor 41 is a device that executes various kinds of functions through the execution of programs. ROM 42 stores a BIOS, etc. RAM 43 is used as a work area of the processor 41.

The auxiliary storage device 44 is configured with, for example, a hard disk device or a semiconductor storage. The auxiliary storage device 44 stores data related to verification of a patient who is prescribed the prescription digital therapeutic app in addition to the prescription digital therapeutic app.

The communication interface 45 is an interface for communicating with other terminal devices or servers via the network 80, or an interface for communicating with surrounding devices. The communication interface 45 complies with Ethernet (registered trademark), Wi-Fi (registered trademark), Bluetooth (registered trademark), Universal Serial Bus (USB), mobile communication systems and other communication standards.

The display 46 is a display, for example, a liquid crystal display or an organic Electro Luminescence (EL).

The input receiving device 47 is, for example, a mouse or a keyboard. Note that, in the case that the patient terminal 40 is a smartphone, an electrostatic capacitance type touch sensor with permeability that does not interfere with the visibility of the image displayed on the display 46 is used as the input receiving device 47. The device which is combined with this type touch sensor and display is called a touch panel.

Note that, not shown, a camera may be provided in the patient terminal 40.

### <Screen of Patient Terminal>

In the exemplary embodiments, the patient terminal 40 displays a content which encourages the patient to continue improving the patient's lifestyle at a time when a predetermined period of time elapses since the patient starts using the prescription digital therapeutic app.

Herein, a time when continuation of improving the patient's lifestyle is likely to be difficult may be set as the time when the predetermined period of time elapses since the patient starts using the prescription digital therapeutic app. In addition, a time when the patient's motivation for his/her appropriate lifestyle lowers may be set as the time when the predetermined period of time elapses since the patient starts using the prescription digital therapeutic app. Further, a time prior to the time when the patient's motivation for his/her appropriate lifestyle lowers may be set as the time when the predetermined period of time elapses since the patient starts using the prescription digital therapeutic app. Herein, the time when therapy phase changes is illustrated as the time when the patient's motivation for his/her appropriate lifestyle lowers. In addition, the time prior to the time when the patient's motivation for his/her appropriate lifestyle lowers may be, for example, the predetermined number of days prior to the time when the patient's motivation for his/her appropriate lifestyle lowers.

Hereinafter, the time when 12, 16, 18 and 20 weeks elapse since the patient starts using the prescription digital therapeutic app are described as examples of the time when a predetermined period of time elapses since the patient starts using the prescription digital therapeutic app.

In addition, in the exemplary embodiments, if the predetermined condition is satisfied at a time when a predetermined period of time elapses since the patient starts using the prescription digital therapeutic app, the patient terminal 40 displays a content which encourages the patient to continue improving the patient's lifestyle.

Herein, the predetermined condition may be the condition that the patient does not perform the behavior which infers continuation of improving the patient's lifestyle. A condition that the measurement of blood pressure value or input of review is not performed by a patient for a period of time will be used as an example of such condition below. In particular, if the condition that the measurement of blood pressure value or input of review is not performed by a patient for a period of time is satisfied at a time when 16 weeks elapse since the patient starts using the prescription digital therapeutic app, the patient terminal 40 displays a content which encourages the patient to continue improving the patient's lifestyle.

Alternatively, in the exemplary embodiments, if the predetermined condition is satisfied when the predetermined period of time elapse since the patient starts using the prescription digital therapeutic app, the patient terminal 40 displays the first content as a content which encourages the patient to continue improving the patient's lifestyle. On the other hand, if the predetermined condition is not satisfied when the predetermined period of time elapses since the patient starts using the prescription digital therapeutic app, the patient terminal 40 displays the second content as a content which encourages the patient to continue improving the patient's lifestyle.

Herein, the predetermined condition may be a condition that a period when an event which is likely to obstruct continuation of improving the patient's lifestyle occurs is approaching. A condition that a long vacation is approaching will be used as an example of such condition below. In particular, if that kind of condition is satisfied at a time when 18 weeks elapse since the patient starts using the prescription digital therapeutic app, the patient terminal 40 displays the first content which encourages the patient to continue improving the patient's lifestyle. On the other hand, if that kind of condition is not satisfied at a time when 18 weeks elapse since the patient starts using the prescription digital therapeutic app, the patient terminal 40 displays the second content which encourages the patient to continue improving the patient's lifestyle.

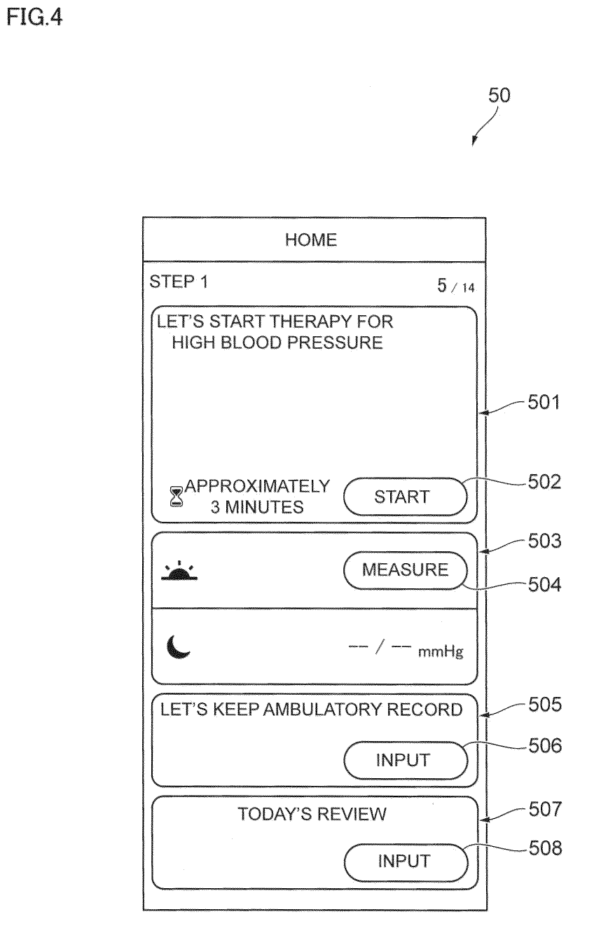

FIG. 4 shows an example of a normal home screen 50 which a patient terminal 40 displays. The normal home screen 50 is a home screen which is displayed at times other than when 12, 16, 18 and 20 weeks elapse since the patient starts using the prescription digital therapeutic app. In addition, even when 12 weeks elapse since the patient starts using the prescription digital therapeutic app, if the patient does not take medicine, the normal home screen 50 is displayed. Furthermore, even when 16 weeks elapse since the patient starts using the prescription digital therapeutic app, if measurement of blood pressure value and input of review are performed by the patient for a period of time, the normal home screen 50 is displayed.

As shown, the normal home screen 50 includes a normal content start region 501, a blood pressure value measurement region 503, an ambulatory record input region 505 and a review input region 507.

The normal content start region 501 is a region for starting the display of a normal content that is an interactive content that makes the patient recognize general things he/she should pay attention to for high blood pressure. For example, the patient taps the “start” button 502 in the normal content start region 501, the patient terminal 40 displays the normal content.

The blood pressure value measurement region 503 is a region for the patient to measure a blood pressure value. For example, the patient taps the “measure” button 504 in the blood pressure value measurement region 503, the patient terminal 40 displays a message for instructing the blood pressure machine to turn on and measure blood pressure. Thus, when the patient measures blood pressure by the blood pressure machine, the patient terminal 40 acquires the blood pressure value from the blood pressure machine, for example, by Bluetooth (registered trademark), and displays the blood pressure value in the blood pressure value measurement region 503. In this case, the blood pressure value measured in the morning is displayed in the upper part of the blood pressure value measurement region 503, and the blood pressure value measured at night is displayed in the lower part of the blood pressure value measurement region 503. Alternatively, the blood pressure value measurement region 503 may be a region for the patient to manually input the blood pressure value measured by the blood pressure machine.

The ambulatory record input region 505 is a region for inputting ambulatory record, which is a record of ambulatory practice that was received for therapy of high blood pressure. In the ambulatory record, there is a record related to such as prescription of medication that a doctor performed in ambulatory practice, a promise which the patient exchanged with the doctor. For example, the patient taps the “input” button 506 in the ambulatory record input region 505, the patient terminal 40 displays an option or an input field for inputting the ambulatory record to enable the patient to input the ambulatory record.

The review input region 507 is a region that the patient input today's review, in other words, what he/she feels about improving lifestyle by reviewing today. The review may be, for example, written by form such as a diary. For example, the patient taps the “input” button 508 in the review input region 507, the patient terminal 40 displays an input field for inputting the review to enable the patient to input the review.

In contrast, the patient terminal 40 displays a home screen to prevent the patient from slump in improving his/her lifestyle at a time when 12, 16, 18 or 20 weeks elapse since the patient starts using the prescription digital therapeutic app.

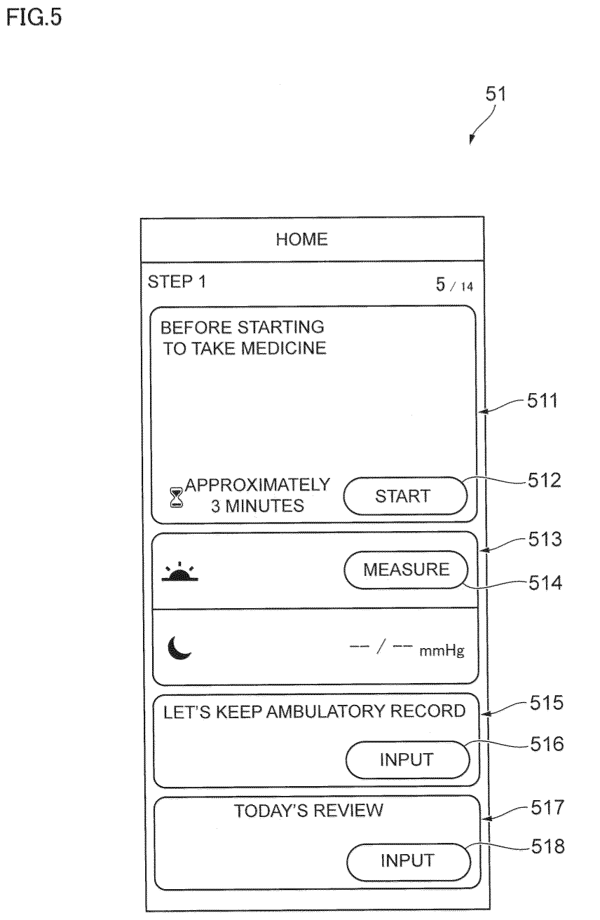

FIG. 5 shows an example of the 12th week home screen 51 which a patient terminal 40 displays. The 12th week home screen 51 is a screen which is displayed if the patient takes medicine at a time when 12 weeks elapse since the patient starts using the prescription digital therapeutic app.

As shown, the 12th week home screen 51 includes a 12th content start region 511, a blood pressure value measurement region 513, a ambulatory record input region 515 and a review input region 517.

The 12th content start region 511 is a region for starting the display of the 12th week content that is an interactive content that makes the patient recognize things he/she should pay attention to when taking medicine. Here, it is assumed that medicine is prescribed to a patient whose high blood pressure does not improve even after 12 weeks elapse since he/she starts using the prescription digital therapeutic app, therefore, when the patient takes medicine at this time, the display of the 12th content will begin. For example, the patient taps the “start” button 512 in the 12th week content start region 511, the patient terminal 40 displays the 12th week content.

The blood pressure value measurement region 513, the ambulatory record input region 515 and the review input region 517 are similar to the blood pressure value measurement region 503, the ambulatory record input region 505 and the review input region 507 in FIG. 4, so description is omitted.

Note that, the determination of whether the patient takes medicine at a time when displaying the 12th week home screen 51 may be performed, based on the ambulatory record which is input in the ambulatory record input region 515.

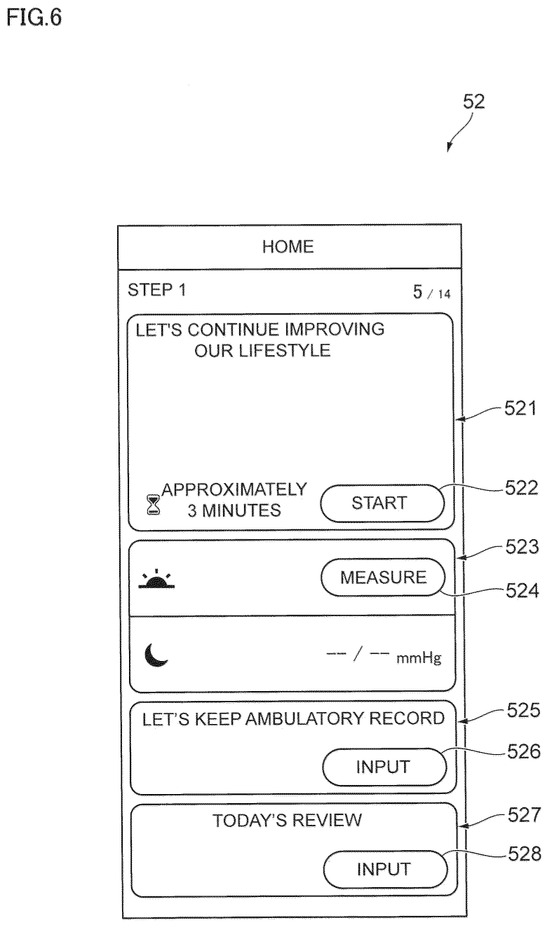

FIG. 6 shows an example of the 16th week home screen 52 which the patient terminal 40 displays. The 16th week home screen 52 is a screen which is displayed if measurement of blood pressure value or input of review is not performed by the patient for a period of time at a time when 16 weeks elapse since patient starts using the prescription digital therapeutic app.

As shown, the 16th week home screen 52 includes a 16th content start region 521, a blood pressure value measurement region 523, an ambulatory record input region 525 and a review input region 527.

The 16th week content start region 521 is a region for starting display of the 16th week content which is an interactive content that makes the patient recognize a necessity of continuing improving lifestyle. For example, the patient taps the “start” button 522 in the 16th week content start region 521, the patient terminal 40 displays the 16th week content.

The blood pressure value measurement region 523, the ambulatory record input region 525 and the review input region 527 are similar to the blood pressure value measurement region 503, the ambulatory record input region 505 and the review input region 507 in FIG. 4, so description is omitted.

Note that, the determination of whether the patient does not perform measurement of blood pressure value for a period of time when displaying the 16th week home screen 52 may be performed, based on the date and time when the blood pressure value is measured in the blood pressure value measurement region 523. In addition, the determination of whether the patient does not perform input of a review for a period of time, when displaying the 16th week home screen 52 may be performed, based on the date and time when the review is input the review input region 527.

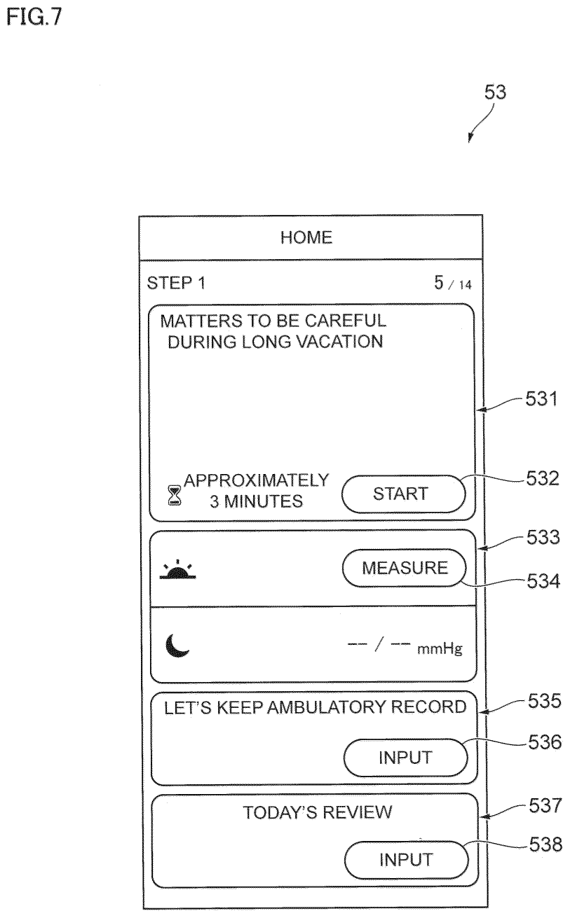

FIG. 7 shows an example of the 18th week first home screen 53 which a patient terminal 40 displays. The 18th week first home screen 53 is a screen which is displayed at a time when 18 weeks elapse since the patient starts using the prescription digital therapeutic app, and at a time when a long vacation is approaching.

As shown, the 18th week first home screen 53 includes a 18th first content start region 531, a blood pressure value measurement region 533, an ambulatory record input region 535 and a review input region 537.

The 18th week first content start region 531 is a region for starting the 18th week first content which is an interactive content that makes the patent recognize the things he/she should pay attention to in order to continue improving lifestyle when he/she spends a long vacation. For example, the patient taps the “start” button 532 in the 18th week first content start region 531, the patient terminal 40 displays the 18th week first specific content.

The blood pressure value measurement region 533, the ambulatory record input region 535 and the review input region 537 are similar to the blood pressure value measurement region 503, the ambulatory record input region 505 and the review input region 507 in FIG. 4, so description is omitted.

Note that, the determination of whether a long vacation is approaching when displaying the 18th week first home screen 53 may be performed by confirming that the current date corresponds to a predetermined date prior to the long vacation. Examples of predetermined dates prior to long vacations are April 15˜April 30, July 20˜August 10, September 1˜September 20, and December 1˜December 31, but these are merely examples. Dates other than these may be added, or some of these dates may be excluded.

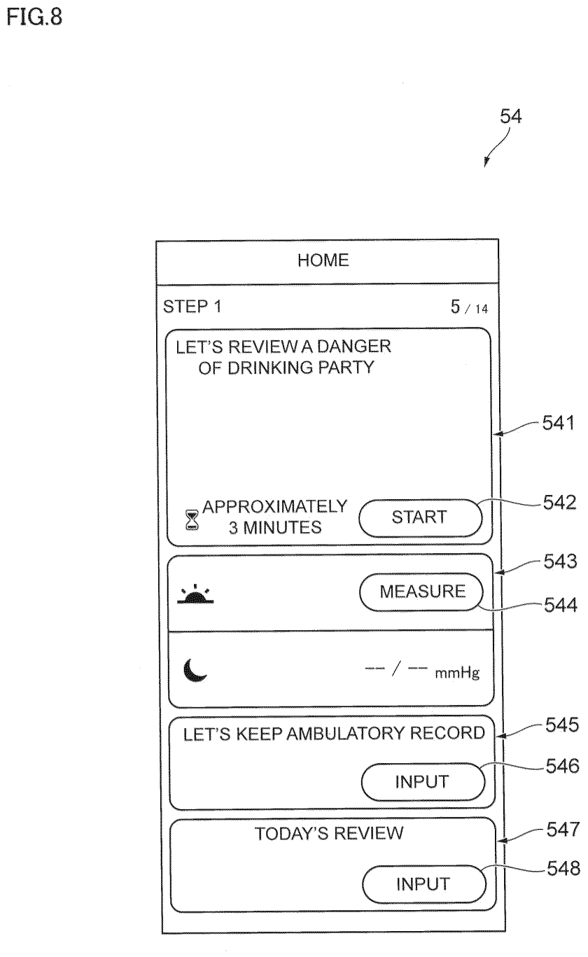

FIG. 8 shows an example of the 18th week second home screen 54 which a patient terminal 40 displays. The 18th week second home screen 54 is a screen which is displayed at a time when 18 weeks elapse since the patient starts using the prescription digital therapeutic app and at a time except for when a long vacation is approaching.

As shown, the 18th week second home screen 54 includes a 18th second content start region 541, a blood pressure value measurement region 543, an ambulatory record input region 545 and a review input region 547.

The 18th week second content start region 541 is a region for starting the display of the 18th week second content that is an interactive content that reminds the user of the dangers of drinking party that may be held even besides long vacations in order to continue improving lifestyle. For example, the patient taps the “start” button 542 in the 18th week second content start region 541, the patient terminal 40 displays the 18th week second specific content.

The blood pressure value measurement region 543, the ambulatory record input region 545 and the review input region 547 are similar to the blood pressure value measurement region 503, the ambulatory record input region 505 and the review input region 507 in FIG. 4, so description is omitted.

Note that, the determination of whether or not a time when a long vacation is approaching when displaying the 18th week second home screen 52 may be performed by checking that the current date does not correspond to predetermined date prior to the long vacation. The predetermined date prior to the long vacation is the same as that described for FIG. 7, so description is omitted.

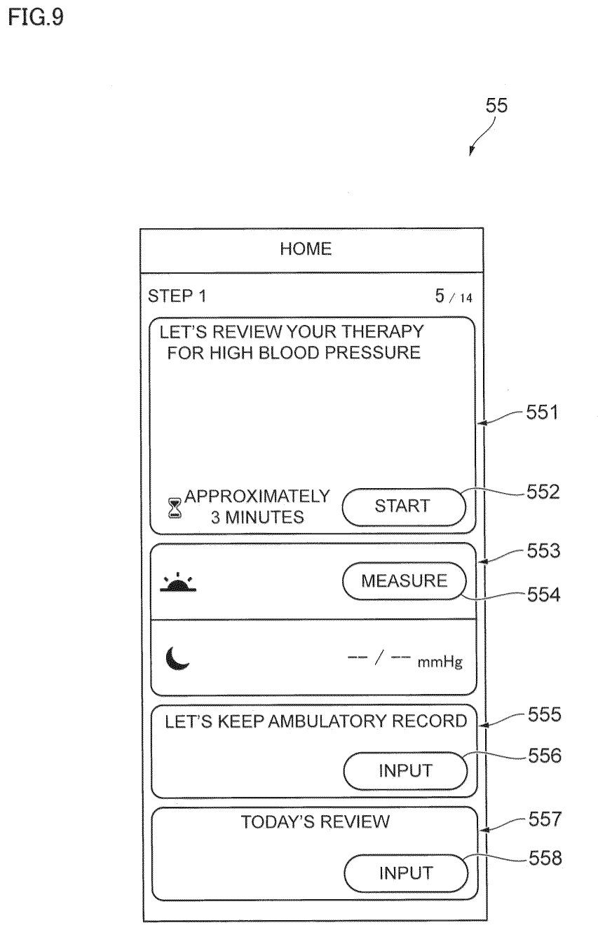

FIG. 9 shows an example of the 20th week home screen 55 which a patient terminal 40 displays. The 20th week home screen 55 is a screen which is displayed at a time when twenty weeks elapse since patient starts using the prescription digital therapeutic app.

As shown, the 20th week home screen 55 includes a 20th content start region 551, a blood pressure value measurement region 553, an ambulatory record input region 555 and a review input region 557.

The 20th week content start region 551 is a region for starting the 20th week content which is an interactive content that encourages reviewing therapy for high blood pressure so far. For example, the patient taps the “start” button 552 in the 20th week content start region 551, the patient terminal 40 displays the 20th content.

The blood pressure value measurement region 553, the ambulatory record input region 555 and the review input region 557 are similar to the blood pressure value measurement region 503, the ambulatory record input region 505 and the review input region 507 in FIG. 4, so description is omitted.

Note that, in the review input region 557, it is possible for the patient to review their use of prescription digital therapeutic app so far and input his/her subjective degree of lifestyle change. This patient's subjective degree of lifestyle change includes, for example, how much the frequency of exercise improves and how much the degree of salt reduction improves. In addition, when the patient's subjective degree of lifestyle change is transmitted to the prescription digital therapeutic app operation server 10, the prescription digital therapeutic app operation server 10 may transmit this patient's subjective degree of lifestyle change to the doctor terminal 30. In general, the user's subjective degree of lifestyle change may be transmitted to the terminal of a mentor for the improvement of the user's lifestyle. In this case, the 20th week home screen 55 is an example of a content configured to allow the user to input information related to the user's lifestyle improvement. In addition, the transmission of patient's subjective degree of lifestyle change to the doctor terminal 30 is an example of which the information related to the improvement of the user's lifestyle is notified to the terminal of the mentor for the improvement of the user's lifestyle.

Note that, in the above, 12, 16, 18 and 20 weeks are indicated as predetermined periods, however, these are merely examples, and this is not limited to the above. However, in case that plural periods of time are set as the time when a predetermined period of time elapses within a set period of improvement, from a view to prevent slump, the plural periods of time may be set in the second half of the set period of improvement more than the first half of the set period of improvement. In the example of FIG. 4 to FIG. 9 also, the insurance medical therapy period is set as 6 months, in other words, 26 weeks, thus 12 weeks are set in the first half of the insurance medical therapy period, and 16, 18 and 20 weeks are set in the second half of the insurance medical therapy period. Therefore, the plural periods of time is set in the second half of the insurance medical therapy period more than the first half of the insurance medical therapy period.

In addition, in the above, as the predetermined condition, the condition that the patient does not perform the behavior which infers the continuation of improving the patient's lifestyle, and the condition that the period when an event which is likely to obstruct continuation of improving the user's lifestyle occurs is approaching are indicated, however, this is not limited to the above. The predetermined condition may be, for example, the condition that the patient's measurement value does not satisfy reference which infers continuation of improving the user's lifestyle, or the condition that the patient is in an environment where it is likely to be difficult for the patient to continue improving the user's lifestyle.

Further, in the above, whether the condition that measurement of blood pressure value or input of review is not performed by the patient for a period of time is satisfied at a time when 16 weeks elapse since the patient starts using the prescription digital therapeutic app is satisfied is determined. Alternatively, whether the condition that a long vacation is approaching is satisfied at a time when 18 weeks elapse since the patient starts using the prescription digital therapeutic app is satisfied is determined. However, it may be decided freely to determine whether which of the predetermined condition is satisfied at a time when certain number of weeks elapse since patient starts using the prescription digital therapeutic app.

Furthermore, in the above, the patient terminal 40 switches whether displaying the content which encourages the patient to continue improving the patient's lifestyle according to whether the condition that measurement of blood pressure value or input of review is not performed by the patient for a period of time is satisfied. Alternatively, the patient terminal 40 switches whether displaying the first content or the second content as a content which encourages the patient to continue improving the patient's lifestyle according to whether the condition that a long vacation is approaching is satisfied. However, the patient terminal 40 may switch any of them according to whether a certain condition is satisfied. In other words, the patient terminal 40 may switch whether displaying a content which encourages the patient to continue improving the patient's lifestyle, and switch whether the first content or the second content as a content which encourages the patient to continue improving the patient's lifestyle.

### <Feature Configuration of Prescription Digital Therapeutic App Operation Server 10>

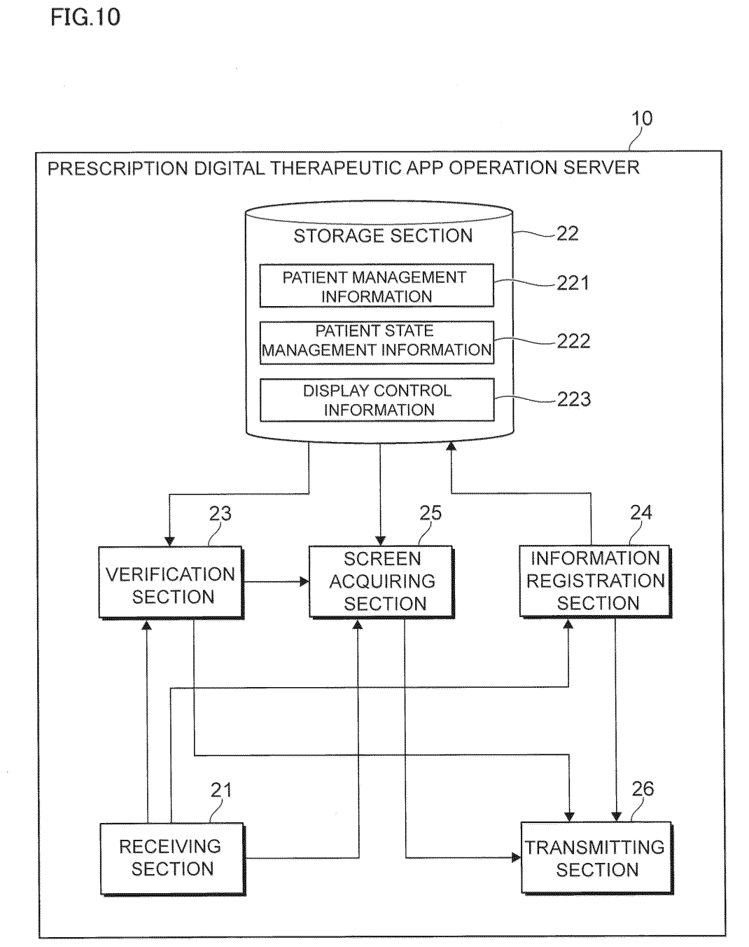

FIG. 10 shows a functional configuration example of a prescription digital therapeutic app operation server 10 in the exemplary embodiments.

As shown, the prescription digital therapeutic app operation server 10 includes a receiving section 21, a storage section 22, a verification section 23, an information registration section 24, a screen acquiring section 25 and a transmitting section 26.

The receiving section 21 receives a patient ID and a prescription code from the patient terminal 40 when the patient performs activation of the prescription digital therapeutic app. In addition, the receiving section 21 receives an account ID and a password from the patient terminal 40 when the patient registers the account ID and the password used for logging in the prescription digital therapeutic app. Further, the receiving section 21 receives patient information from the patient terminal 40 when the patient registers information related to the patient (hereinafter referred to as “patient information”).

The receiving section 21 receives the account ID and the password from the patient terminal 40 when the patient logs in the prescription digital therapeutic app. In addition, the receiving section 21 receives a request starting an interactive content from the patient terminal 40 when the patient requests starting an interactive content. Further, the receiving section 21 receives information obtained by measurement or input (hereinafter referred to as “measurement input information”) from the patient terminal 40 when the patient performs measurement or input on the home screen.

The storage section 22 stores patient management information 221 that manages the patient, patient state management information 222 that manages state of the patient and display control information 223 to control a display of the home screen for the patient terminal 40. The patient management information 221, the patient state management information 222 and the display control information 223 are described below.

The verification section 23 verifies the patient when performing activation of the prescription digital therapeutic app, based on the patient ID and the prescription code, which the receiving section 21 receives from the patient terminal 40. As described below, the patient ID and the prescription code are registered in the patient management information 221 stored in the storage section 22, therefore, the verification section 23 refers the patient management information 221 when this verification is performed. In addition, the verification section 23 verifies the patient when using the prescription digital therapeutic app, based on the account ID and the password, which the receiving section 21 received from the patient terminal 40. As described below, the account ID and the password are registered in the patient management information 221 stored in the storage section 22, therefore, the verification section 23 refers the patient management information 221 when this verification is performed.

Information registration section 24 registers the account ID and the prescription code, which the receiving section 21 receives from the patient terminal 40, in the patient management information 221 stored in the storage section 22. In addition, the information registration section 24 registers patient information, which the receiving section 21 received from the patient terminal 40, in the patient management information 221 stored in the storage section 22. Further, the information registration section 24 regards a day when the receiving section 21 received patient information as a start day of use of the prescription digital therapeutic app, and registers the start day in the patient management information 221 stored in the storage section 22. In this case, the process which the information registration section 24 performs is an example of a process that causes the user to start using an application which supports improvement of the user's lifestyle over a set period of improvement. Furthermore, the information registration section 24 registers measurement input information that the receiving section 21 received, in the patient state management information 222 stored in the storage section 22.

The screen acquiring section 25 acquires a home screen to be displayed on the patient terminal 40 when the verification section 23 verifies the patient based on the account ID and the password, and the verification is successful. Specifically, the screen acquiring section 25 acquires the home screen according to the number of weeks since the patient starts using the prescription digital therapeutic app and whether the conditions specified for that number of weeks are satisfied. For example, if the number of weeks since the start of use of the prescription digital therapeutic app and the conditions defined for that number of weeks are defined in the display control information 223 stored in the storage section 22, as described below, the screen acquiring section 25 refers to this display control information 223 and acquires the home screen. Alternatively, the screen acquiring section 25 may acquire the home screen by program logic without reference to the display control information 223. Note that, the screen acquiring section 25 may display the home screen based only on the number of weeks since the user starts using the prescription digital therapeutic app. In this case, the process performed by the screen acquiring section 25 is an example of the process of displaying a content that encourages the user to continue improving the user's lifestyle at a time when a predetermined period of time elapses since the user starts using the application.

The screen acquiring section 25 acquires the interactive content which is displayed on the patient terminal 40, when the receiving section 21 receives the request starting interactive content from the patient terminal 40.

The transmitting section 26 transmits the result of verification to the patient terminal 40 when verification for the patient is performed at the time when the verification section 23 performs activation of the prescription digital therapeutic app, based on the patient ID and the prescription code. In addition, the transmitting section 26 transmits the result of verification to the patient terminal 40 when the verification section 23 performs verification for the patient at the time when the prescription digital therapeutic app is used, based on the account ID and the password.

The transmitting section 26 transmits notification of completion of registration to the patient terminal 40 when the information registration section 24 registers the account ID and the password in the patient management information 221. In addition, the transmitting section 26 transmits notification of completion of registration to the patient terminal 40 when the information registration section 24 registers the patient information and the start day of use in the patient management information 221. Further, the transmitting section 26 transmits notification of completion of registration to the patient terminal 40 when the information registration section 24 registers measurement input information in the patient state management information 222.

When the screen acquiring section 25 acquires the home screen, the transmitting section 26 transmits the home screen to the patient terminal 40. In addition, when the screen acquiring section 25 acquires the interactive content, the transmitting section 26 transmits the interactive content to the patient terminal 40.

Note that, in the above, in the prescription digital therapeutic app operation system 1, the patient terminal 40 includes a function of receiving and displaying a screen, and the prescription digital therapeutic app operation server 10 includes all other functions, however, this is not limited to the above. The patient terminal 40 may include some of functions which in the above the prescription digital therapeutic app operation server 10 includes.

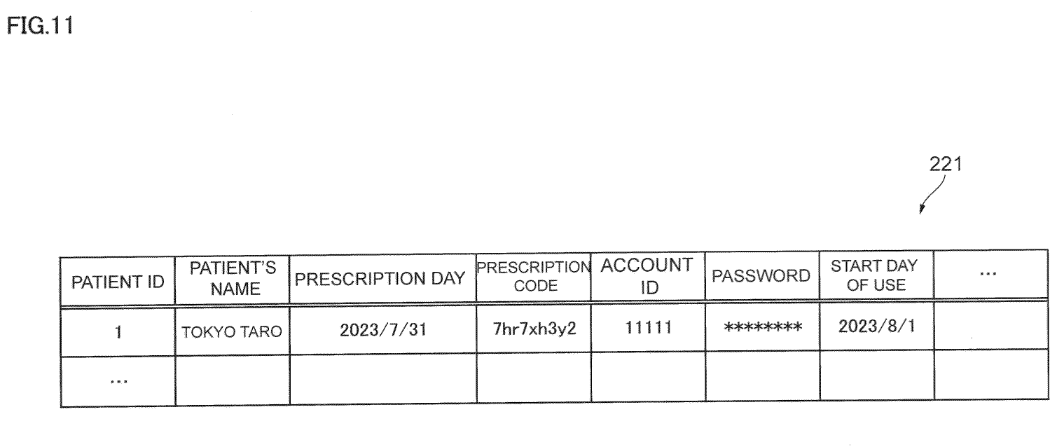

FIG. 11 shows a specific example of the patient management information 221 stored in the storage section 22.

As shown, the patient management information 221 includes a patient ID, a patient's name, a prescription day, a prescription code, an account ID, a password and a start day of the use.

The patient ID is an ID that identifies the patient who is notified from an insurance medical institution at a moment when the prescription digital therapeutic app is prescribed.

The patient's name is registered if the patient's name is notified from the insurance medical institution. In FIG. 11, the patient's name is “Tokyo Taro”.

The prescription day is a day when the prescription digital therapeutic app is prescribed. In FIG. 11, the prescription day is Jul. 31, 2023.

The prescription code is a code that is notified to the patient for activation of the prescription digital therapeutic app. The prescription code is generated every prescription. The limited term of use is determined in the prescription code, and the prescription code which expired the limited term of use becomes invalid. The limited term of use of the prescription code is, for example, 3 days from the prescription day.

The account ID and the password are necessary information when logging in the prescription digital therapeutic app.

The start day of the use is the day when use of the application starts. In FIG. 11, the start day of the use is Aug. 1, 2023.

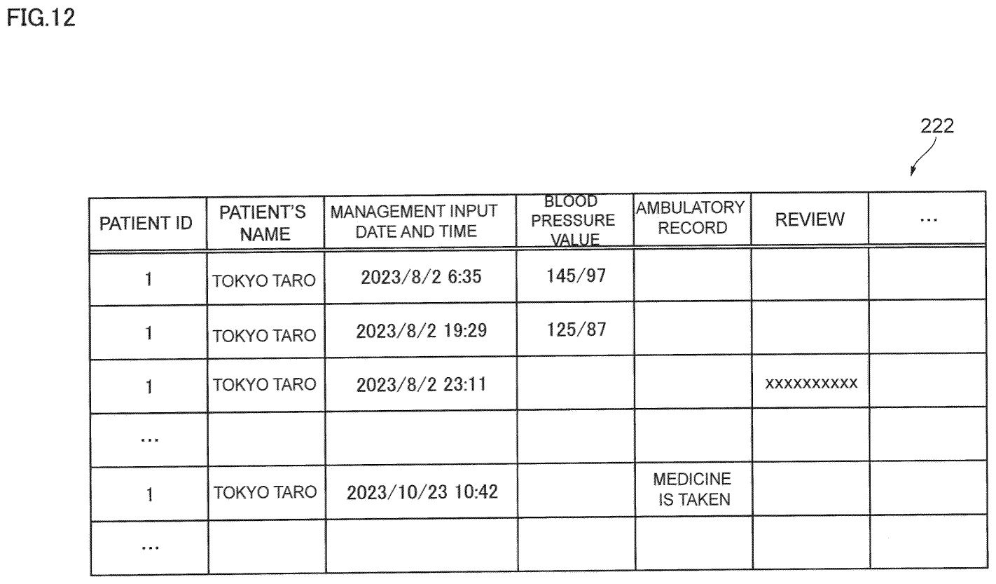

FIG. 12 shows a specific example of the patient state management information 222 stored in the storage section 22.

As shown, the patient state management information 222 includes a patient ID, a patient's name, a measurement input date and time, a blood pressure value, an ambulatory record and a review.

The patient ID and the patient's name are similar to the patient ID. The patient's name is included in the patient management information 221 in FIG. 11. The patient ID is used to link with the patient management information 221.

The measurement input date and time is the date and time when measurement of blood pressure value, inputting ambulatory record or inputting review is performed.

The blood pressure value is the blood pressure value which is measured in a blood pressure value measurement region of the home screen. In FIG. 12, the blood pressure value is measured at 6:35 on Aug. 2, 2023, which is the day after the start day of the use, and it shows that the maximum blood pressure value is “145”, and the minimum blood pressure value is “97” in the same date and time. Note that, the blood pressure value of night on Aug. 2, 2023 are also shown in the patient state management information 222.

The ambulatory record is the ambulatory record which is input at an ambulatory inputting region of the home screen. FIG. 12 shows that stating is input, which is the stating that the patient takes medicine at 10:42 on Oct. 23, 2023, which is the day after 12 weeks from the start day of the use.

The review is a review which is input at a review input region of the home screen. FIG. 12 shows that the review is input at 23:11 on Aug. 2, 2023, which is the day after the start day of the use.

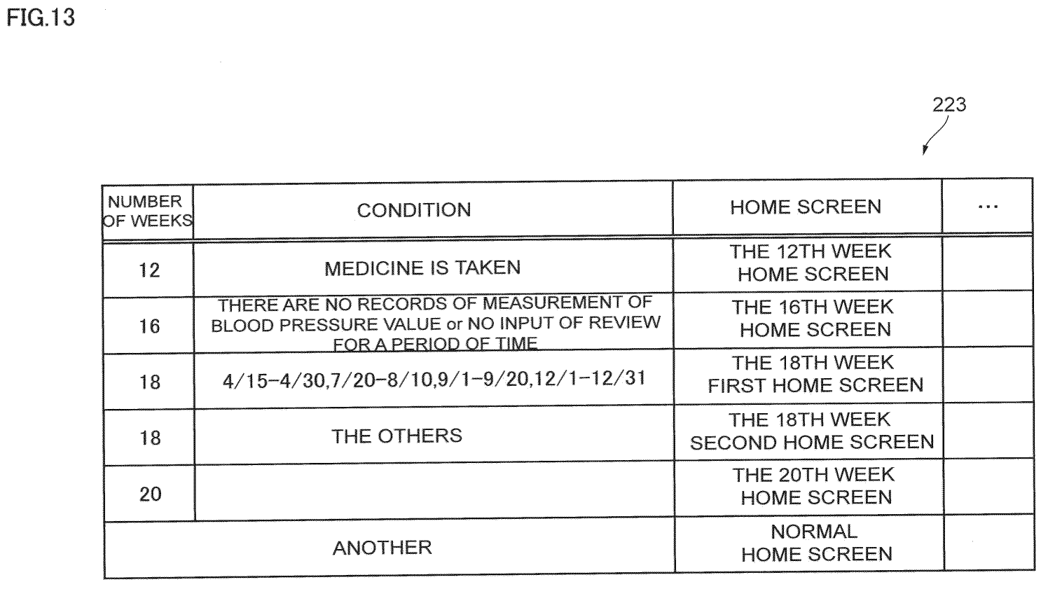

FIG. 13 shows a specific example of the display control information 223 stored in the storage section 22.

As shown, the display control information 223 includes a number of weeks, a condition and a home screen.

The number of weeks is a number of weeks since the patient starts using the prescription digital therapeutic app.

The condition is the condition which is determined for the number of weeks.

The home screen is the home screen which is displayed on the patient terminal 40 if the corresponding condition is satisfied at a time when a corresponding number of weeks elapses since the patient starts using the prescription digital therapeutic app.

By defining shown in FIG. 13, the display of the home screen shown in FIG. 4 to FIG. 9 is performed.

Note that, as described above, this display control information 223 needs not to be provided, when the screen acquiring section 25 acquires the home screen by the program logic.

### <Operations of the Prescription Digital Therapeutic App Operation Server>

Firstly, a doctor determines that it is necessary for a patient to prescript the prescription digital therapeutic app, and requests issue of the prescription code to the prescription digital therapeutic app operation server 10 by operating the doctor terminal 30. Herewith, the prescription digital therapeutic app operation server 10 issues the prescription code and register it in the patient's patient state management information. In addition, the prescription digital therapeutic app operation server 10 transmits the prescription code to the doctor terminal 30, and, for example, the doctor prints the prescription code on a form, and hands the form to the patient.

On the other hand, the patient terminal 40 requests the prescription digital therapeutic app to the prescription digital therapeutic app operation server 10, according to the patient's operation. Herewith, the prescription digital therapeutic app operation server 10 transmits the prescription digital therapeutic app to the patient terminal 40, and the patient installs the prescription digital therapeutic app transmitted in the patient terminal 40. However, the prescription digital therapeutic app cannot be used when just it is installed, and activation by inputting the prescription code is necessary.

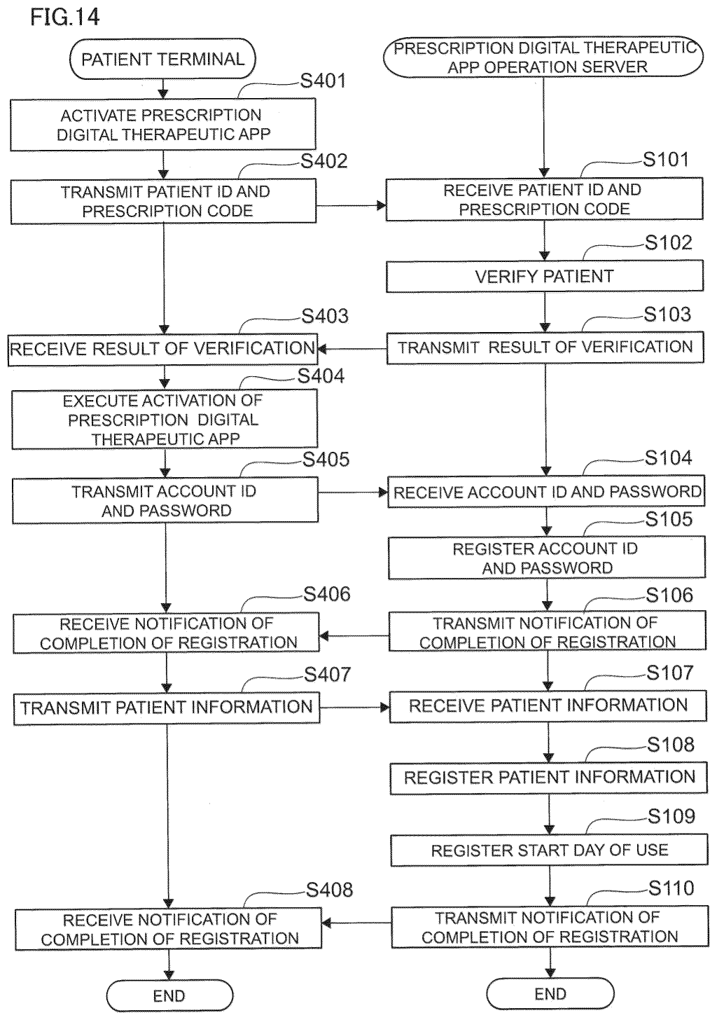

FIG. 14 is a sequence diagram showing an example of operations of the prescription digital therapeutic app operation system 1 when patients register various kinds of information by performing activation of the prescription digital therapeutic app.

As shown, firstly, the patient terminal 40 activates the prescription digital therapeutic app, according to operation by the patient (Step 401). When the prescription digital therapeutic app is activated, the patient terminal 40 transmits a patient ID and a prescription code to the prescription digital therapeutic app operation server 10 (Step 402).

Thereby, in the prescription digital therapeutic app operation server 10, the receiving section 21 receives the patient ID and the prescription code from the patient terminal 40 (Step 101).

Then, the verification section 23 verifies the patient by a reference between the prescription code that the verification section 23 receives with the patient ID in Step 101 and the prescription code linked with the patient ID in the patient management information 221 (Step 102).

In case that these two prescription codes match, the verification section 23 determines that the verification is successful. On the other hand, in case that these two prescription codes do not match, the verification section 23 determines that the verification is failed. Note that, even if two prescription codes match, when the limited term of use of the prescription code expires, the verification section 23 determines that the verification is failed. The limited term of use of the prescription code may be, for example, three days from the prescription day.

In the prescription digital therapeutic app operation server 10, the transmitting section 26 transmits the result of verification to the patient terminal 40 (Step 103).

Thereby, the patient terminal 40 receives the result of verification from the prescription digital therapeutic app operation server 10 (Step 403). In case that the verification is successful, code that permits the activation of the prescription digital therapeutic app is included in the result of verification. Therefore, the patient terminal 40 executes the activation of the prescription digital therapeutic app (Step 404). As a result, it becomes possible to use the prescription digital therapeutic app in the patient terminal 40.

On the other hand, in case that the verification is failed, code that permits the activation of the prescription digital therapeutic app is not included in the result of verification. In this case, an error screen is displayed on the display 46 (refer to FIG. 3) of the patient terminal 40.

When the activation of the prescription digital therapeutic app is performed, it becomes possible to register various kinds of information for the prescription digital therapeutic app operation server 10.

Specifically, the patient terminal 40 transmits the account ID and the password, which are used to log in, to the prescription digital therapeutic app operation server 10 (Step 405).

Thereby, in the prescription digital therapeutic app operation server 10, the receiving section 21 receives the account ID and the password from the patient terminal 40 (Step 104). Thereafter, the information registration section 24 registers the account ID and the password, which are received in Step 104, in the patient management information 221 (Step 105).

When the registration of the account ID and the password, which are used to log in, in the prescription digital therapeutic app operation server 10, the transmitting section 26 transmits a notification of completion of registration to the patient terminal 40 (Step 106).

Thereby, the patient terminal 40 receives the notification of completion of registration from the prescription digital therapeutic app operation server 10 (Step 406).

After the completion of the registration of the account ID and the password, which are used to log in, the patient input the patient information in the patient terminal 40. When the input is fixed, the patient terminal 40 transmits this patient information to the prescription digital therapeutic app operation server 10 (Step 407).

Thereby, in the prescription digital therapeutic app operation server 10, the receiving section 21 receives the patient information from the patient terminal 40 (Step 107). Thereafter, the information registration section 24 registers the patient information, which is received in Step 107, in the patient management information 221 (Step 108). In addition, the information registration section 24 determines that it is possible to use the prescription digital therapeutic app by the registration of this patient information, the information registration section 24 regards the day when the patient information is registered as the start day of the use of the prescription digital therapeutic app, and registers this start day of the use in the patient management information 221 (Step 109).

When the registration of the patient information and the start day of the use is completed, in the prescription digital therapeutic app operation server 10, the transmitting section 26 transmits a notification of completion of registration to the patient terminal 40 (Step 110).

Thereby, the patient terminal 40 receives the notification of completion of registration from the prescription digital therapeutic app operation server 10 (Step 408). The completion of registration is displayed on the display 46 (refer to FIG. 3) of the patient terminal 40.

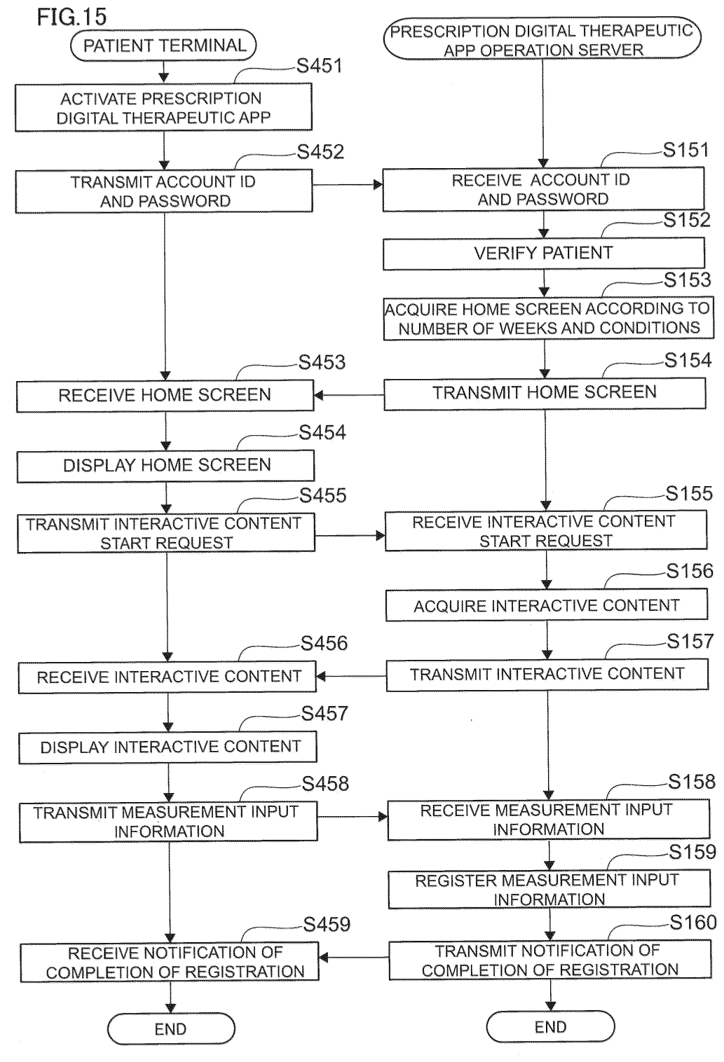

FIG. 15 is a sequence diagram showing an example of operations of the prescription digital therapeutic app operation system 1 when patients use the prescription digital therapeutic app after performing activation of the prescription digital therapeutic app.

As shown, firstly, the patient terminal 40 activates the prescription digital therapeutic app, according to operation by the patient (Step 451). When the prescription digital therapeutic app activates, the patient terminal 40 transmits the account ID and the password, which are used to log in, to the prescription digital therapeutic app operation server 10 (Step 452).

Thereby, in the prescription digital therapeutic app operation server 10, the receiving section 21 receives the account ID and the password from the patient terminal 40 (Step 151).

Then, the verification section 23 performs verifying process by the account ID and the password received in Step 151 (Step 152).

In case that the account ID and the password received in Step 151 are present in the patient management information 221, the verification section 23 determines that the verification is successful. On the other hand, in case that the account ID and the password received in Step 151 are not present in the patient management information 221, the verification section 23 determines that the verification is failed.

In case that the verification is determined to be successful, the screen acquiring section 25 acquires a home screen according to the number of weeks since the start day of use of the prescription digital therapeutic app by the patient and the conditions defined for this number of weeks (Step 153). Specifically, the screen acquiring section 25 firstly calculates the number of days from the start day of use registered in the patient management information 221 in Step 109 of FIG. 14 to the current date, and divides this number of days by 7 to obtain the number of weeks from the start day of use. When the number of days is not divisible by 7, the decimal point may be rounded off, rounded down, or rounded up. Next, the screen acquiring section 25 identifies the record that the number of weeks in the display control information 223 in FIG. 13 is the number of weeks from the start day of use obtained above. Next, the screen acquiring section 25 determines whether the conditions in that record are satisfied. In case that the condition is “Medicine is taken” or “There are no records of measurement of blood pressure value or no input of review for a period of time”, the screen acquiring section 25 refers to the patient state management information 222 in FIG. 12 and determines whether the condition is satisfied. Additionally, in the case that the condition is “4/15˜4/30, 7/20˜8/10, 9/1˜9/20, 12/1˜12/31”, the screen acquiring section 25 refers to the current date and determines whether the condition is satisfied. If it is determined that the condition is satisfied, the screen acquiring section 25 acquires the home screen for that record. Herein, the home screen is any of the home screens shown in FIGS. 4 to 9. Therefore, the home screen includes a content start region to start displaying any of the normal content, 12th week content, 16th week content, 18th week first content, 18th week second content, and 20th week content.

In the prescription digital therapeutic app operation server 10, the transmitting section 26 transmits the home screen acquired in Step 153 to the patient terminal 40 (Step 154).

Thereby, the patient terminal 40 receives the home screen from the prescription digital therapeutic app operation server 10 (Step 453). Then, the patient terminal 40 displays the home screen received in Step 453 on the display 46 (refer to FIG. 3) (Step 454).

Note that, in case that the verification is failed, an error screen is displayed on the display 46 (refer to FIG. 3) of the patient terminal 40.

Thereafter, when the patient requests starting the interactive content on the content start region in the home screen, the patient terminal 40 transmits the request starting the interactive content to the prescription digital therapeutic app operation server 10 (Step 455). Herein, when the home screen is the normal home screen 50, the interactive content is the normal content. When the home screen is the 12th week home screen 51, the interactive content is the 12th week content. When the home screen is the 16th week home screen 52, the interactive content is the 16th week content. When the home screen is the 18th week first home screen 53, the interactive content is the 18th week first content, and when the home screen is the 18th week second home screen 54, the interactive content is the 18th week second content. When the home screen is the 20th week home screen 55, the interactive content is the 20th week content.

Thereby, in the prescription digital therapeutic app operation server 10, the receiving section 21 receives the request starting interactive content (Step 155). Then, the screen acquiring section 25 acquires the interactive content (Step 156).

In the prescription digital therapeutic app operation server 10, the transmitting section 26 transmits the interactive content to the patient terminal 40 (Step 157).

Thereby, the patient terminal 40 receives the interactive content from the prescription digital therapeutic app operation server 10 (Step 456). Then, the patient terminal 40 displays the interactive content received in Step 456 on the display 46 (refer to FIG. 3) (Step 457).

Thereafter, when the patient performs measurement or input on the home screen, the patient terminal 40 transmits the measurement input information obtained by the measurement or input to the prescription digital therapeutic app operation server 10 (Step 458). Herein, as shown in FIG. 4 to FIG. 9, the measurement input information is the blood pressure value measured in the blood pressure value measurement region, the ambulatory record input in the ambulatory record input region, the review input in the review input region or so on.

Thereby, in the prescription digital therapeutic app operation server 10, the receiving section 21 receives measurement input information (Step 158). Then, the information registration section 24 registers measurement input information in the patient state management information 222 (Step 159). Note that, herein, the measurement input information registered in the patient state management information 222 is referred to in step 153 when this flowchart is operated at a later time.

When the registration of the measurement input information is completed, in the prescription digital therapeutic app operation server 10, the transmitting section 26 transmits the notification of the completion of registration to the patient terminal 40 (Step 160).

Thereby, the patient terminal 40 receives the notification of the completion of registration from the prescription digital therapeutic app operation server 10 (Step 459). The completion of registration is displayed on the display 46 (refer to FIG. 3) of the patient terminal 40.

### <Example of Deformation>

In the above exemplary embodiments, an application whose purpose is therapy for high blood pressure is described as an example of an application which supports improvement of the user's lifestyle, however, this is not limited to the above. Application which supports improvement of the user's lifestyle may be an application for the therapy of other diseases that require therapy, lifestyle changes, and lifestyle innovations for a long period of time. Herein, other diseases include post-transplant, anticancer drug treatment, renal dysfunction, dialysis, lifestyle-related diseases in general and so on. Alternatively, the application which supports improvement of the user's lifestyle may be a health care application that aims to change behavior in diet and exercise.

In the above exemplary embodiments, the timing for displaying a content to prevent slowdown is set at the time when 12, 16, 18, and 20 weeks elapse since the start of use of the therapy application, but this is not limited to this time period, however, this is not limited to the above. Appropriate period of time according to the purpose of the application may be used as the timing for displaying content to prevent slump.

For example, when the purpose of the application is the therapy of disease, for returning to therapy purpose, a timing that seemed to be approximately “got used to” such as two or three months or a half year after the start of therapy, such timing may be the timing for displaying content to prevent slump.

In addition, it may be possible to set a time when minor problems due to therapy are likely to occur or when side effects are likely to occur, and to use this set time as the timing for displaying a content to prevent slump. This makes it possible to cope with pregnancy, etc. according to the time, or to cope with side effects that occur according to the time during anticancer drug treatment, etc.

In the above exemplary embodiments, the condition that measurement of blood pressure value or input of review is not performed by patient for a period of time is used as the condition to display content for preventing slump when certain number of weeks have elapsed since the start of use of the prescription digital therapeutic app, however, this is not limited to the above. Appropriate condition according to the purpose of the application may be used as the condition for displaying content to prevent slump.

For example, the condition that the result of therapeutic intervention does not satisfy the reference may be the condition for displaying a content to prevent slump. Here, the result of therapeutic intervention includes weight, blood glucose level and so on, in addition to the blood pressure value described above. In this case, the condition that the results of the therapeutic intervention do not satisfy the reference is an example of the condition that the patient's measured value does not satisfy reference which infers the continuation of improving the patient's lifestyle.

In addition, the condition that the implementation status of the therapeutic intervention does not satisfy the reference may also be used as a condition for displaying a content to prevent slump. Here, the implementation status of the therapeutic intervention includes the implementation status of exercise, and so on, in addition to the implementation status of measurement as described above. Moreover, the condition that the implementation status of the therapeutic intervention does not satisfy the reference may be, for example, the following three conditions. The first condition is the condition that the rate of use of the application by the patient lowers. The second condition is that the rate of performing a predetermined behavior by the patient with respect to a target lowers. The third condition is the number of characters in a diary related to improvement of the lifestyle which the patient input is decreasing. Herein, the diary may be, for example, the review input in the review input region 507 to 557 in FIG. 4 to FIG. 9, and the number of characters in a diary may be, for example, the number of characters in a free input in a diary. In this case, the condition that the implementation status of the therapeutic intervention does not satisfy the reference is an example of the condition that the patient does not perform the behavior which infers the continuation of improving the patient's lifestyle.

Further, a condition related to the external environment, such as seasonal events, temperature, weather and so on may also be used as a condition for displaying content to prevent slump. For example, for a patient who is known to reside in a region with heavy snowfall based on geographical data, when the condition related to the external environment indicates winter season, such as content indicating measures to be taken if the patient is unable to exercise outdoors, may be presented as content to prevent slump. In this case, the condition related to external environment is an example of the condition that the patient is in an environment where it is easily to become difficult for the patient to continue improving the patient's lifestyle.

Furthermore, a condition according to the acceptance process or behavior change stage may also be used as a condition displaying content to prevent slump.

The acceptance process is the idea that the process of deep grief progresses through five emotional stages which are denial, anger, bargaining, depression, and acceptance. The condition for displaying can be that the patient's psychological state in response to the therapeutic intervention is in a predetermined state. In other words, the content would be presented in accordance with the acceptance process, based on numerical and emotional expressions.

The behavior change stages are these stages in a model which considers that when a person changes behavior, he/she goes through five stages which are indifference stage, interest stage, preparation stage, implementation stage, and maintenance stage. In order to move ahead through the stages, it is necessary to understand which stage the person is currently in and work with them according to each stage, which means presenting a content according to the stage.

### Effect of the Exemplary Embodiments

In the exemplary embodiments, the content that encourages the user to continue improving his/her lifestyle is displayed at a time when a predetermined period of time elapses since the user starts using the application that supports the improvement of his/her lifestyle. Thereby, it is possible to intensively display the content that encourages the user to continue improving his/her lifestyle at a time when the user is likely to experience so-called “slump,” is likely to occur, therefore, it is an effective proposal related to improving the lifestyle.

In addition, in the exemplary embodiments, at a time when a predetermined period of time elapses since the user starts using the application that supports improvement of lifestyle, the content that encourages the user to continue improving the user's lifestyle is displayed if, for example, there are factors that obstruct continuation of improving lifestyle. Thereby, even if, for example, there are factors that obstruct continuation of improving lifestyle, it became effectively possible to continue improving lifestyle or to implement behavior changes.

### <Processor>

In the exemplary embodiments, the processor refers to a processor in a broad sense including, in addition to a general-purpose processor (for example, a Central Processing Unit (CPU)), a dedicated processor (for example, a Graphical Processing Unit (GPU), an Application specific Integrated Circuit (ASIC), a Field Programmable Gate Array (FPGA), a program logic device, etc.).

Moreover, the operation of the processor in the exemplary embodiments may not only be performed by a single processor, but may be also performed in cooperation by multiple processors which are present in physically far position. In addition, the order of execution of each operation in the processor is not limited to the order described in the exemplary embodiments, but may be changed.

### <Program>

The process, which the prescription digital therapeutic app operation server 10 in the exemplary embodiments performs is prepared, for example, as a program of application software and so on.

In this case, the program which executes the exemplary embodiments is regarded as a program that causes a computer to executes causing a user to start using an application which supports improvement of the user's lifestyle over a set period of improvement, and displaying a content which encourages the user to continue improving the user's lifestyle at a time when a predetermined period of time elapses since the user starts using the application.

Note that, the program which executes the exemplary embodiments can be provided not only by the communication means, but also can be provided by storing a recording medium such as CD-ROM.

### <Conclusion>

The disclosure examples described in the above-described exemplary embodiments are shown below.

(((1)))

An information processing system including at least one processor, wherein the at least one processor cause user to start using an application which supports improvement of the user's lifestyle over a set period of improvement, and displays a content which encourages the user to continue improving the user's lifestyle at a time when a predetermined period of time elapses since the user starts using the application.

According to the information processing system, it is possible to encourage the user to continue improving the user's lifestyle at a time when a predetermined period of time elapses since the user starts using the application that supports improvement of lifestyle.

(((2)))

The information processing system described in (((1))), wherein the time when the predetermined period of time elapses is a period of time when continuation of improving the user's lifestyle is likely to be difficult.

According to the information processing system, it is possible to encourage the user to continue improving the user's lifestyle at a time when continuation of improving the user's lifestyle is likely to be difficult.

(((3)))

The information processing system described in (((1))), wherein the time when the predetermined period of time elapses is a period when the user's motivation for his/her appropriate lifestyle lowers or a period prior to the period when the user's motivation lowers.

According to the information processing system, it is possible to encourage the user to continue improving the user's lifestyle when the user's motivation for his/her appropriate lifestyle lowers or a period prior to the period when the user's motivation lowers.

(((4)))

The information processing system described in (((1))), wherein plural periods of time is set as the time when the predetermined period of time elapses, and the plural periods of time is set in a second half of the set period of improvement more than a first half of the set period of improvement.

According to the information processing system, it is possible to encourage the user to continue improving the user's lifestyle intensively in the second half of the set period of improvement rather than the first half of the set period of improvement.

(((5)))

The information processing system described in (((1))), wherein the at least one processor displays the content if a predetermined condition is satisfied when the predetermined period of time elapses.

According to the information processing system, it is possible to switch whether it encourages the user to continue improving the lifestyle, according to whether the predetermined condition is satisfied when the predetermined period of time elapses since the user starts using the application that supports improvement of lifestyle.

(((6)))

The information processing system described in (((1))), wherein the at least one processor displays a first content as the content if a predetermined condition is satisfied when the predetermined period of time elapses, and displays a second content as the content if the predetermined condition is not satisfied when the predetermined period of time elapses.

According to the information processing system, it is possible to switch how to encourage the user to continue improving the lifestyle, according to whether the predetermined condition is satisfied when the predetermined period of time elapses since the user starts using the application that supports improvement of lifestyle.

(((7)))

The information processing system described in (((5))) or (((6))), wherein the predetermined condition is a condition that the user does not perform a behavior which infers continuation of improving the user's lifestyle.

According to the information processing system, it is possible to switch whether it encourages the user to continue improving the lifestyle or how to encourage the user to continue improving the lifestyle, according to whether the user does not perform the behavior which infers the continuation of improving the user's lifestyle.

(((8)))

The information processing system described in (((7))), wherein the predetermined condition is a condition that a rate of use of the application by the user lowers.

According to the information processing system, it is possible to switch whether it encourages the user to continue improving the lifestyle or how to encourage the user to continue improving the lifestyle, according to whether the rate of use of the application by the user lowers.

(((9)))

The information processing system described in (((7))), wherein the predetermined condition is a condition that a rate of performing a predetermined behavior by the user with respect to a target lowers.

According to the information processing system, it is possible to switch whether it encourages the user to continue improving the lifestyle or how to encourage the user to continue improving the lifestyle, according to whether the rate of performing a predetermined behavior by the user with respect to a target lowers.

(((10)))

The information processing system described in (((7))), wherein the predetermined condition is a condition that a number of characters in a diary related to improvement of the user's lifestyle which the user inputs lowers.

According to the information processing system, it is possible to switch whether it encourages the user to continue improving the lifestyle or how to encourage the user to continue improving the lifestyle, according to whether the number of characters in a diary related to improvement of the user's lifestyle which the user inputs lowers.

(((11)))

The information processing system described in (((5))) or (((6)) wherein the predetermined condition is a condition that the user's measurement value does not satisfy reference which infers continuation of improving the user's lifestyle.

According to the information processing system, it is possible to switch whether it encourages the user to continue improving the lifestyle or how to encourage the user to continue improving the lifestyle, according to whether the user's measurement value does not satisfy reference which infers the continuation of improving the user's lifestyle.

(((12)))

The information processing system described in (((5))) or (((6))), wherein the predetermined condition is a condition that a period when an event which is likely to obstruct continuation of improving the user's lifestyle occurs is approaching.

According to the information processing system, it is possible to switch whether it encourages the user to continue improving the lifestyle or how to encourage the user to continue improving the lifestyle, according to whether the period when an event which is likely to obstruct continuation of improving the user's lifestyle occurs is approaching.

(((13)))

The information processing system described in (((5))) or (((6))), wherein the predetermined condition is a condition that the user is in an environment where it is likely to be difficult for the user to continue improving the user's lifestyle.

According to the information processing system, it is possible to switch whether it encourages the user to continue improving the lifestyle or how to encourage the user to continue improving the lifestyle, according to whether the user is in an environment where it is likely to be difficult for the user to continue improving the user's lifestyle.

(((14)))

The information processing system described in (((1))), wherein the content is configured to enable the user to input information related to the improvement of the user's lifestyle.

According to the information processing system, it is possible for the user to input the information related to the improvement of the lifestyle on the content.

(((15)))

The information processing system described in (((14))), wherein the information related to the improvement of the user's lifestyle is notified to a terminal of a mentor for the improvement of the user's lifestyle.

According to the information processing system, it is possible to notify the mentor of lifestyle improvement of the information related to the improvement of the user's lifestyle.

(((16)))

An information processing method including causing a user to start using an application which supports improvement of the user's lifestyle over a set period of improvement, and displaying a content which encourages the user to continue improving the user's lifestyle at a time when a predetermined period of time elapses since the user starts using the application.

According to the information processing method, it is possible to encourage the user to continue improving the user's lifestyle at a time when a predetermined period of time elapses since the user starts using the application.

Note that, in this program, it is possible to combine technologies corresponding to (((2))) to (((15))).

(((17)))

A non-transitory computer-readable medium storing a program that causes a computer to execute; causing a user to start using an application which supports improvement of the user's lifestyle over a set period of improvement, and displaying a content which encourages the user to continue improving the user's lifestyle at a time when a predetermined period of time elapses since the user starts using the application.

According to the program, it is possible to encourage the user to continue improving the user's lifestyle at a time when a predetermined period of time elapses since the user starts using the application.

Note that, in this program, it is possible to combine technologies corresponding to (((2))) to (((15))).

The foregoing description of the exemplary embodiment of the present invention has been provided for the purposes of illustration and description. It is not intended to be exhaustive or to limit the invention to the precise forms disclosed. Obviously, many modifications and variations will be apparent to practitioners skilled in the art without departing from the scope and spirit of the present invention. The exemplary embodiment was chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. An information processing system comprising:
at least one processor, wherein
the at least one processor causes a user to start using an application which supports improvement of the user's lifestyle over a set period of improvement, and
displays a content which encourages the user to continue improving the user's lifestyle at a time when a predetermined period of time elapses since the user starts using the application.

2. The information processing system according to claim 1, wherein the time when the predetermined period of time elapses is a period of time when continuation of improving the user's lifestyle is likely to be difficult.

3. The information processing system according to claim 1, wherein the time when the predetermined period of time elapses is a time when the user's motivation for his/her appropriate lifestyle lowers or a time prior to the time when the user's motivation lowers.

4. The information processing system according to claim 1, wherein a plurality of periods of time is set as the time when the predetermined period of time elapses, and
the plurality of periods of time is set in a second half of the set period of improvement more than a first half of the set period of improvement.

5. The information processing system according to claim 1, wherein the at least one processor displays the content if a predetermined condition is satisfied when the predetermined period of time elapses.

6. The information processing system according to claim 1, wherein the at least one processor displays a first content as the content if a predetermined condition is satisfied when the predetermined period of time elapses, and displays a second content as the content if the predetermined condition is not satisfied when the predetermined period of time elapses.

7. The information processing system according to claim 5, wherein the predetermined condition is a condition that the user does not perform a behavior which infers continuation of improving the user's lifestyle.

8. The information processing system according to claim 7, wherein the predetermined condition is a condition that a rate of use of the application by the user lowers.

9. The information processing system according to claim 7, wherein the predetermined condition is a condition that a rate of performing a predetermined behavior by the user with respect to a target lowers.

10. The information processing system according to claim 7, wherein the predetermined condition is a condition that a number of characters in a diary related to improvement of the user's lifestyle which the user inputs lowers.

11. The information processing system according to claim 5, wherein the predetermined condition is a condition that the user's measurement value does not satisfy reference which is infers continuation of improving the user's lifestyle.

12. The information processing system according to claim 5, wherein the predetermined condition is a condition that a period when an event which is likely to obstruct continuation of improving the user's lifestyle occurs is approaching.

13. The information processing system according to claim 5, wherein the predetermined condition is a condition that the user is in an environment where it is likely to be difficult for the user to continue improving the user's lifestyle.

14. The information processing system according to claim 1, wherein the content is configured to enable the user to input information related to the improvement of the user's lifestyle.

15. The information processing system according to claim 14, wherein the information related to the improvement of the user's lifestyle is notified to a terminal of a mentor for the improvement of the user's lifestyle.

16. An information processing method comprising:
causing a user to start using an application which supports improvement of the user's lifestyle over a set period of improvement, and
displaying a content which encourages the user to continue improving the user's lifestyle at a time when a predetermined period of time elapses since the user starts using the application.

17. A non-transitory computer-readable medium storing a program that causes a computer to execute;
causing a user to start using an application which supports improvement of the user's lifestyle over a set period of improvement, and
displaying a content which encourages the user to continue improving the user's lifestyle at a time when a predetermined period of time elapses since the user starts using the application.
